(19) United States
(12) Patent Application Publication
(10) Pub. No.: US 2026/0014256 A1
(43) Pub. Date: Jan. 15, 2026

(54) DOUBLE EMULSION STRUCTURE FOR LOW FREQUENCIES ULTRASOUND-TRIGGERED DRUG DELIVERY

(71) Applicants: SORBONNE UNIVERSITE, PARIS (FR); CENTRE NATIONAL DE LA RECHERCHE SCIENTIFIQUE, PARIS (FR); INSTITUT NATIONAL DE LA SANTE ET DE LA RECHERCHE MEDICALE, Paris (FR); ECOLE NORMALE SUPERIEURE, PARIS (FR); UNIVERSITE PARIS CITE, PARIS (FR)

(72) Inventors: Nicolas TAULIER, Paris (FR); Wladimir URBACH, Paris (FR); Chloë THIMONIER, Paris (FR)

(21) Appl. No.: 19/139,635

(22) PCT Filed: Dec. 13, 2023
(86) PCT No.: PCT/EP2023/085666

§ 371 Date: Jun. 16, 2025

(30) Foreign Application Priority Data
Dec. 16, 2022 (WO) PCT/IB2022/000727

(51) Int. Cl. A61K 41/00; A61K 9/00; A61K 9/113; A61K 31/7105; A61K 31/711; A61K 38/16; A61K 47/14; C07K 16/00

(52) U.S. Cl. CPC A61K 41/0028; A61K 9/0009; A61K 9/113; A61K 31/7105; A61K 31/711; A61K 38/16; A61K 47/14; C07K 16/00

(57) ABSTRACT

The present invention relates to methods and drug delivery systems for targeted drug delivery, and more particularly for low frequencies ultrasound-triggered drug delivery.

The present invention relates to methods and drug delivery systems for controlled drug delivery, and more particularly for low frequencies ultrasound-triggered drug delivery.

Controlled drug delivery has been studied as a solution to increase the efficacy of treatment while decreasing toxicity and side effects. It consists in encapsulating an active in an object (capsule, shell etc) and delivering it in a controlled way at the target site. The drug is protected from multiple physiological barriers which degrade it and a higher percentage reaches the target. At the site of interest, the particles can then accumulate passively (Enhanced Permeability and Retention effect) or actively. When the particles reach the target site, they can be destabilized through an external stimulus to deliver their content abruptly or to induce a diffusion of the drug from the particle to the surrounding environment. Liposomes have been widely studied to serve such a function, and few have been commercialized (e.g. Doxil, Onivyde).

A main disadvantage of liposomes is that they are leaky before reaching the target location.

Other encapsulation methods include polymeric nanoparticles, which are difficult to produce in high-throughput or micelles which can only contains lipophilic drugs.

Another encapsulation method consists in using emulsions of perfluorocarbon (PFC) oils. PFC's are biocompatible, immiscible with water or organic oils, and have the particular ability to undergo a phase change when stimulated with ultrasound. Indeed, when treated with an acoustic pressure above a threshold, liquid PFC droplets become gaseous bubbles. This process is called Acoustic Droplet Vaporization (ADV). Since PFC oils are hydrophobic and lipophobic they can act as a shell surrounding a water or oil core in which a drug can be solubilized. The phase change resulting from ADV is thought to be the mechanism responsible for the ultrasound-triggered delivery of actives from PFC emulsions. A drawback of this mechanism is that the acoustic pressure may also induce the implosion of the bubble (called inertial cavitation) which is a violent mechanism that can induce deleterious local side effects of the surrounding tissues in addition to altering the encapsulated drug, especially if the drug is a protein such as an antibody.

It has recently been shown that ultrasound can be used to trigger a diffusion mechanism, without vaporization or thermal effects, when using non-perfluorocarbonated oils that cannot vaporize (N. Al Rifai et al., Journal of Materials Chemistry B 8 (8) (2020) 1640-1648). While this article mentions the possibility of avoiding ADV release it is only directed to vehicle hydrophobic bioactive compounds while in the field of controlled bioactive molecule, there is still a need to be able to deliver hydrophobic and/or hydrophilic bioactive molecules.

Surprisingly, the Inventors have now been able to prepare a new formulation allowing the targeted and local release of any kind of compounds, in particular water-soluble, with ultrasounds.

The present invention relates to a double emulsion structure composed of a water-soluble compound comprised in at least one water-based droplet wherein said water-based droplet is included in an oily droplet and wherein said structure allows the on-demand release of said water soluble compound when subjected to low-frequency ultrasound, such release advantageously occurs without compromising the oil droplet stability.

In other words, the present invention relates to a double emulsion structure composed of at least one water based droplet that comprises at least one first biocompatible amphiphilic molecule and at least one water soluble compound, wherein said water based droplet is included in an oily droplet that comprises at least one biocompatible oil and at least one second biocompatible amphiphilic molecule and wherein said structure allows the on-demand release of said water soluble compound when subjected to low frequency ultrasound.

Accordingly, the double emulsion structure is composed of oily droplets of a size comprised between 100 and 1000 nm; and said oily droplets comprises water based droplets of a size comprised between 50 and 800 nm.

Size of the droplets may be assessed as described in part 1.5.3. of the Example.

The oily droplets are composed of a water phase (i.e. water-based droplets) that represents between 10 and 80% of the total volume of the oily droplets; accordingly, the oily phase of the oily droplets represents between 20 and 90% of the total volume of the oily droplets. Such ratio between oil and water phases is chosen depending on the compounds to be formulated in the double emulsion structure; for example, in case only water-soluble compounds are formulated, the water phase is increased (for example, from 60 up to 80% of the total volume of the oily droplets). In case the compounds to be formulated are a mixture of water-soluble compounds and lipophilic compounds, then the ratio between oil and water phases may be about 50:50.

The water-based droplets are composed of saline solution, at least one first biocompatible amphiphilic molecule and at least one water-soluble compound that is preferably a therapeutic active agent.

Preferably, the saline solution has the plasma osmotic pressure and has a concentration of NaCl in water of approximately 0.5% to 1.5% wt.

The first biocompatible amphiphilic molecule may be selected in the group consisting of lipids, proteins (e.g. Serum albumin), polymers or surfactants (e.g. dendrimers like Dendri-TAC, oligomers like FiTACn, such as F₈TAC₁₃, or HiTACn, TPGS 1000, TPGS 750M, Tween 20, Tween 80, amino acid or sugar derived surfactants), in the last case surfactant has an HLB (hydrophilic-lipophilic balance) comprised between 3 and 6.

The oily droplets comprise at least one biocompatible oil and at least one second biocompatible amphiphilic molecule.

The second biocompatible amphiphilic molecule may be selected in the group consisting of lipids, proteins (e.g. Serum albumin), polymers or surfactants (e.g. dendrimers like Dendri-TAC, oligomers like FiTACn, such as F₈TAC₁₃, or HiTACn, TPGS 1000, TPGS 750M, Tween 20, Tween 80, amino acid or sugar derived surfactants) where the surfactant last case has a HLB (hydrophilic-lipophilic balance) comprised between 8 and 18.

The surface area of droplets should be filled with biocompatible amphiphilic molecules to ensure their stability and to avoid coalescence. The number of amphiphilic molecules needed to fill the droplet surface area is calculated by dividing the latter by the surface area of an amphiphilic molecule. The last value can be derived from the measurement of interfacial tension. The technique consists of measuring the interfacial tension of a millimetric water droplet in bulk oil, or alternatively a millimetric oil droplet in bulk water for various concentration of amphiphilic molecules solubilized either in oil or water (depending on their solubilization). For instance, the technique of the spinning droplet method can be used (for example with the following device: https://www.directindustry.fr/prod/kruess-gmbh/product-14849-1772556.html) or of the sessile and pendant droplet methods. The interfacial tension value will increase as the surfactant concentration increases, until it reaches a plateau at the critical micelle concentration (CMC). Below CMC, most amphiphilic molecules are located at the water/oil interface. By dividing the surface of the millimetric droplet (derived from the droplet size) by the number of amphiphilic molecules, the surface area of one amphiphilic molecule can be obtained.

According to a first embodiment, said at least one biocompatible oil is a non-vaporizable perfluorocarbon (PFC) oil, such as perfluorohexane, perfluoro-octyl bromide, perfluoro-15-crown-5-ether and the second biocompatible amphiphilic molecule is fluorinated surfactant such as telomeric FTAC surfactant (for instance F₈TAC₁₃, composed of a fluorocarbon chain made of 8 carbons and a repetition of 13 telomers), dentriTAC surfactants.

Advantageously, when prepared with PFC, no passive diffusion of the water-soluble compound from the double emulsion structure occurs, the release of the water-soluble compound being only obtained when the double emulsion structure is subjected to low frequency ultrasounds. This property is very interesting for therapeutic application as there is no loss of active agent.

According to a second embodiment, said at least one biocompatible oil is selected from the group comprising mono-, di- or glycerol triesters; derived molecules of glycerol, mono-, di- or tri- or tetra-esters of citric acid; derived molecules from citric acid; fatty acids; acid monoesters fat; steroids; sphingolipids; glycerophospholipids; polyketics; saccharolipids; terpenes; lipids derived from prenol; essential oils; grease substitutes; waxes (triglycerides); and combinations of these abovementioned oil compound. Preferred examples of such oils are tributyl-o-acetyl citrate (ATBC).

The double emulsion structure according to present invention is such that the oily droplets comprise one or several water-based droplets.

According to a particular embodiment, the therapeutical active agent is a chemical compound, a protein, a peptide, an antibody a DNA structure or an RNA structure.

According to a particular embodiment, the therapeutic agent is comprised in the water-based droplet of the double emulsion structure of the invention.

According to another particular embodiment, at least two therapeutic agents are comprised in the same or each in different water-based droplets.

According to another further embodiment, the oily droplet contains another therapeutical active agent.

That is to say that the double emulsion structure may be composed of:
oily droplets that do not contain any active agent and that contain only one water-based droplet; said water-based droplet may contain one or several water soluble therapeutic active agents; or
oily droplets composed of non-perfluorocarbonated oil that contain at least one hydrophobic therapeutic active agent and that contain only one water-based droplet; said water-based droplet may contain one or several water soluble therapeutic active agents; or
oily droplets that do not contain any active agent and that contain several water-based droplets, each water based droplet comprising one or several soluble therapeutic active agent; according to one specific embodiment, each water-based droplet contains one soluble therapeutic active agent but said soluble therapeutic active agent may be different from one water-based droplet to the other; or
oily droplets composed of non-perfluorocarbonated oil that contain at least one hydrophobic therapeutic active agent and that contain several water-based droplets, each water-based droplet comprising one or several soluble therapeutic active agents; according to one specific embodiment, each water based droplet contains one soluble therapeutic active agent but said soluble therapeutic active agent may be different from one water based droplet to the other.

According to a particular embodiment, the double emulsion structure of the invention is lyophilized, that is to say in a dry form, substantially free of the water that is not encapsulated. Lyophilization is well known by the person skilled in the art, it may be conducted as described in “Perfluorocarbon nanodroplets as potential nanocarriers for brain delivery”. C. Bérard, et al. Pharmaceutics 14 (2022) 1498.

Such dry form of the double emulsion structure may be directly administered to a subject, for example via topical, oral, intranasal route or combinations thereof. The dry form of the double emulsion structure also allows long-term storage before use in liquid form, e.g., for intravenous administration, after the addition of water or an aqueous solution.

According to another particular embodiment, the double emulsion structure of the invention is in an aqueous continuous phase.

In such an embodiment, the present invention relates to a double emulsion for low frequencies ultrasound-triggered drug delivery comprising:
an aqueous continuous phase;
a discontinuous phase of oily droplets comprising water-based droplets as previously defined.

Advantageously, the double emulsion structure according to the invention can release the at least one therapeutic active agent when it is subject to ultrasounds at a frequency of 0.5 to 2 MHz over an insonation time comprised between 0.1 to 30 minutes; preferably, when the therapeutic active agent is water soluble, the insonation time is comprised between 0.1 and 5 minutes and when the therapeutic active agent is hydrophobic, the insonation time is comprised between 0.1 and 20 minutes.

The present invention thus also relates to a method for the on-demand release of a water soluble compound, such as a therapeutic agent, comprising subjecting a double emulsion structure according to the invention, to an acoustic signal at a frequency of 0.5 to 2 MHz over an insonation time comprised between 0.1 to 30 minutes; preferably, the insonation time is comprised between 0.1 and 5 minutes and, when the double emulsion structure comprises also hydrophobic therapeutic active agent, the insonation time is comprised between 0.1 and 20 minutes.

Preferably, the maximum peak negative pressures of the ultrasounds (expressed in absolute value) to release the content of the droplets are between 0.2 and 1 MPa.

By “on-demand release of a compound”, it is understood that the double emulsion structure of the invention is able to release by induced diffusion said compound when subjected to an acoustic signal and that such release stops when said acoustic signal is interrupted progressively.

Such control of the release of the compounds contained in the double emulsion structure with acoustic signal allows for targeting very particular area in the body of a subject (organ or tissue); it is thus very advantageous for therapeutic treatment as the therapeutic active agents may be released only where they are needed, limiting loss of therapeutic active agent and possible deleterious effects without therapeutic benefit.

The present invention thus relates to the double emulsion structure of the invention for use as a medicament.

There is no limitation in the choice of the therapeutic active agent that is selected depending on the disease to be treated. For example, the therapeutic active agent may be an anticancer, anti-inflammatory, antioxidant, antithrombotic, antibiotic, antibacterial, antiviral, antifungal, antiparasitic drug, or a combination thereof.

The therapeutic use of the double emulsion structure comprises administering it to a living organism; it may be by intravenous route, oral route, topical route, intranasal route, pulmonary route, trans-mucosal route, or combinations thereof.

According to one or more embodiments, the double emulsion structure is administered intravenously.

The formulation of the double emulsion structure of the invention is chosen depending on its route of administration.

For example, in addition to the dry and liquid forms, the double emulsion structure may be incorporated into implantable gels, such as subcutaneous gels; into cutaneous patches or dressings; into sprayable formulations etc. . . . .

In a particular embodiment, the oily droplets of the emulsion further comprise receptor-specific ligands of the organism into which the double emulsion structure is administered. In this way, control of drug transport in the body and/or targeting of tissues or organs of interest such as, for example, tumors, sites of infection or inflammation, can be enhanced. preferably, the ligands are hydrophilic and may be bound to amphiphilic molecule encapsulating the oily droplet, at the surface of the oily droplets.

The present invention further relates to a process of preparation of a double emulsion structure of the invention wherein said double emulsion structure is generated in at least one microfluidic device using flow-focusing geometries. An example of such a process is detailed in part 1.3. of the example.

The present invention also relates to a kit for treating a patient subject to a disease comprising a double emulsion structure according to the invention and a focused ultrasound device (FUS).

### FIGURES

FIG. 1—Scheme of all droplet types used. A: Multicore PFH double emulsions. B: Single-core PFH double emulsions. C: Multi-core ATBC emulsions. D: Plain ATBC emulsions. Droplets A-C are used to carry a hydrophilic cargo, droplet D is used to carry a hydrophobic cargo.

FIG. 2—A: Schematic representation of the flow-focusing junction used to produce plain ATBC single emulsion droplets.

The ATBC dispersed (pink) phase arrives at the level of the nozzle and is pinched by the continuous phase to produce monodisperse droplets. The nozzle width is 40 μm. All channels are 20 μm deep and 100 μm wide. B: Schematic representation of the production steps of multi-core (PFH or ATBC) double emulsions. The core aqueous phase and the oil phase are sonicated to produce the primary nanoemulsion. C: Schematic representation of two-nozzle device made of two flow-focusing junctions (1-2-3 & 3-4-5) used to produce the single-core double emulsion. The channel between the two junctions (channel 3) is hydrophobic. The flow rates were adjusted to encapsulate exactly one water droplet in each double emulsion. All channels are 50 μm deep and 100 μm wide. The first nozzle (at junction 1-2-3) is 35 μm and the second one (at junction 3-4-5) is 70 μm.

FIG. 3—A: Release of fluorescent probe from PFH and ATBC droplets. normalized by R³ and nⱼ, where R is the radius and n_{f} is the number of droplets in the focal zone of the transducer, plotted against acoustic energy. The meaning of the symbols is summarized on the right part of the figure: The first digit specifies the radius of the droplet, the second the number of periods in the signal, MC indicates the multi-core droplets, then PFH or ATBC indicate the oil used to make the droplet. The data for the 20 μm radius droplets fitted with eq. (2) lead to β=5.33×10⁻⁷ μm⁻³ (+/−6.05×10⁻⁸) and α=0.686 (+/−0.006).

Black dot: 20 μm 5c MC PFH; white and black dot: 20 μm 20c MC PFH; white dot: 20 μm 50c MC PFH; black square: 20 μm 5c ATBC; white square: 20 μm 5c ATBC; downside white triangle: 30 μm 5c ATBC; upside black triangle: 20 μm 5c MC ATBC and upside white triangle: 20 μm 50c MC ATBC.

B: Release from 30 μm multi-core PFH (MC PFH) shown as empty blue circles and plain ATBC (ATBC) droplets shown as empty blue squares, treated with 50-cycle pulses, fitted with eq. 1 with β=8.85×10⁻⁷ μm⁻³ (+/−3.52×10⁻⁸) and α=0.71 (+/−0.03).

White dot: 30 μm 50c MC PFH and white square: 30 μm 50c ATBC.

FIG. 4—Release of fluorescent probe from single-core PFH droplets, normalized by R³ and n_{f}, where R is the radius and n_{f} is the number of droplets in the focal zone, plotted versus the acoustic energy. 20 μm radius droplets are plotted as filled triangles, 30 μm droplets are plotted as empty triangles, droplets treated with 5-cycle pulses are shown in red and droplets treated with 50-cycle pulses are shown in blue. β=8.47×10⁻⁷ μm⁻³ (+/−5.38×10⁻⁸) and α=1.60 (+/−0.03).

Black dot: 20 μm 5c SC PFH; white dot: 20 μm 50c SC PFH; black square: 30 μm 5c SC PFH and white square: 30 μm 50c SC PFH.

### EXAMPLES

The following example demonstrates that perfluoro-hexane (PFH) and tributyl-o-acetyl citrate (ATBC) emulsions can release their content without ADV, at high acoustic frequency.

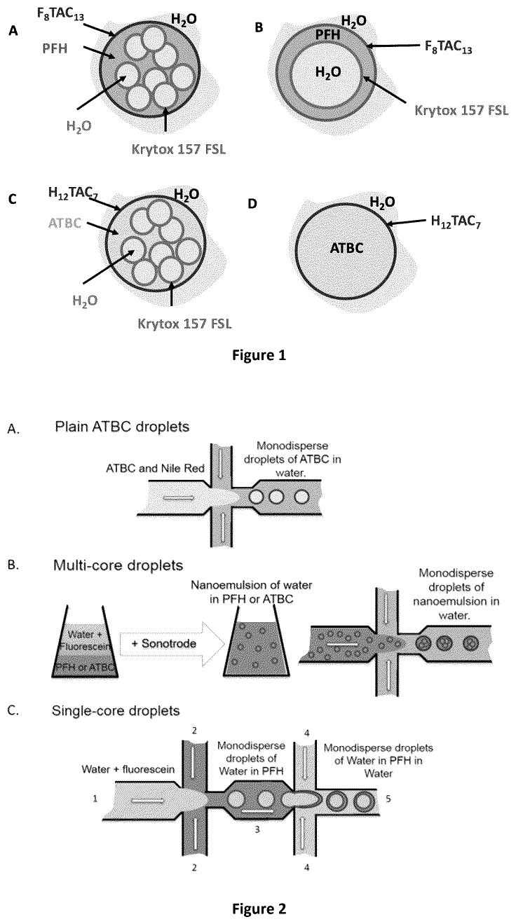

The release of sodium fluorescein, a hydrophilic fluorescent probe, from three types of water core double emulsion droplets is assessed (FIG. 1):
multiple water nanodroplets, in PFH, in water, referred to as multi-core PFH droplets,
a single water microdroplet in PFH in water, referred to as single-core PFH droplets,
and multiple water nanodroplets, in ATBC, in water, referred to as multi-core ATBC droplets.

The release of Nile red, a hydrophobic fluorescent probe, from single emulsions of ATBC in water, referred to as plain ATBC droplets has also been studied.

### I. MATERIALS AND METHODS

### I.1. Materials

Unless otherwise stated, all chemicals were filtered with 0.2 μm pore size Acrodisc Syringe Filters (from Pall, France) and used without further purification. All aqueous solutions were made using Milli-Q IQ 7000 Type-1 water Purification System. PDMS Sylgard 184 was purchased from Neyco (France). Photoresist SU8 was purchased from Chimie Tech Services (France). Silicon wafers were obtained from BT Electronics (France). The MFCS-EZ, pressure controllers used to infect the fluids into the microfluidic chips and the flow units (sizes S and M) used to measure the flow rates were purchased from Fluigent (Le Kremin-Bicêtre, France). The 0.35 mm diameter biopsy puncher was bought from World Precision Instruments (UK), while the plasma cleaner was purchased from Harrick Scientific (NY, United States).

Perfluorohexane and the fluorinated surfactant, Krytox FSL 157, were purchased respectively from ABCR GmbH (Germany) and Costenoble (Germany). The surfactants F₈TAC₁₃ and H₁₂TAC₇ are homemade (see below).

Nile red was purchased from Merck (France) and sodium fluorescein was obtained from VWR (France).

Tributyl O-acetylcitrate, methanol and NaCl were obtained from Sigma Aldrich (France).

I.2. Synthesis of F₈TAC₁₃ and H₁₂TAC₇Surfactants

F-TAC and H-TAC surfactants are amphiphilic molecules composed of two structural units:

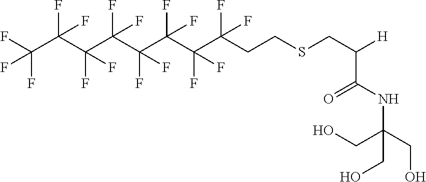

One of the units is a water-soluble oligomer of Tris(hydroxymethyl) aminomethane (Tris) acrylamide units, which constitutes the polar head of the surfactant. Depending on the conditions carried out for their synthesis, it is possible to tune the average number of Tris-acrylamide units.

The second unit is a fluorinated tail (in the case of FTAC) or hydrocarbonated tail (in the case of H-TAC) that acts as a fluorophilic or hydrophobic anchorage ensuring the stabilization of the PFC or ATBC droplet. The F-TAC used in the current study is made of a perfluoro-octyl tail endowed with thirteen Tris-acrylamide units (F₈TAC₁₃). The H-TAC used is made of a dodecane tail endowed with seven Tris-acrylamide units.

### I.3. Microfluidic Devices for Droplet Production

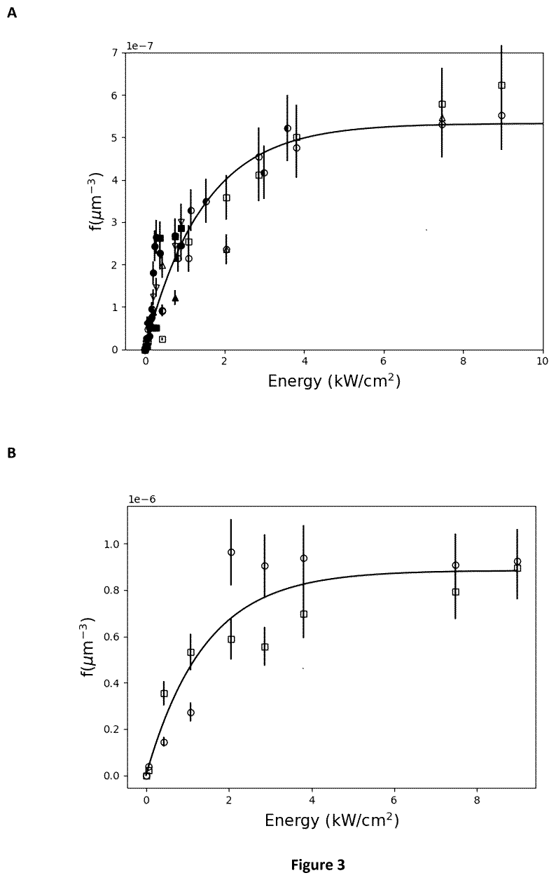

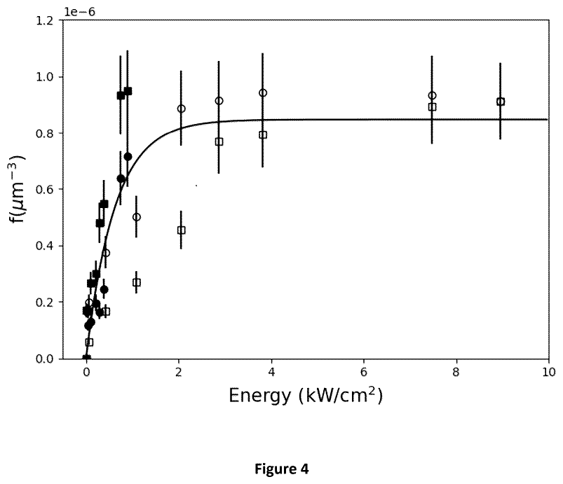

The droplets were generated in microfluidic devices using flow-focusing geometries (FIG. 2). Two types of chips have been manufactured.

The first one, suitable for the production of plain ATBC single emulsions and multi-core double emulsion droplets (FIGS. 2A and B), uses a single flow-focusing junction. All channels are 20 μm deep, 100 μm wide, and the nozzle size is 20 μm.

The second type of chip was used for the production of single-core PFH droplets and is made of two flow-focusing junctions in series inside one chip, as shown in FIG. 2C. All channels are 50 μm deep and 100 μm wide, the nozzle sizes are 35 μm (first nozzle between sections 1-2-3 on FIG. 2C) and 70 μm (second nozzle between sections 3-4-5 on FIG. 2C). Both chips were first designed on AutoCAD. They were printed on a wafer using a two-photon polymerization printer, a Nanoscribe GT Photonic Professional device, with a negative-tone photoresist IP-S (Nanoscribe GmbH, Germany) and 25× objective, directly on silicon substrates after nitrogen plasma cleaning. To reduce printing time, a shell writing strategy was applied. It consists in fabricating a dense shell delimiting the structure, the inner part being only partly polymerized in the form of a scaffold.

After developing in propylene glycol methyl ether acetate (PGMEA) for 30 min and isopropanol (5 min), a batch polymerization is performed with UV-exposure. The microfluidic chips were made using polydimethylsiloxane (PDMS) and its curing agent at a ratio of 10:1 poured onto the wafer which served as a mold for the circuit. It was degassed in a vacuum and then baked at 70° C. for 2 h. The inlets and outlets were punched with a 0.35 mm diameter biopsy puncher. The chips were cleaned with isopropanol and dried with nitrogen gas.

### I.4. Surface Treatment

### I.4.1. Multi-Core and Plain Droplets

The single flow-focusing chip is used for single emulsions and multi-core droplets underwent a hydrophilic surface treatment. The circuit side of the chip and a microscope glass slide were both activated in an air plasma (18 W for 1 min). They were then put in contact to bond, and placed in the oven at 70° C. for 30 min to strengthen the bonding.

The bonded chip was cooled to room temperature and activated again in an air plasma for 1 min. Water was inserted inside the chip with Fluigent pressure controllers to make the circuit walls hydrophilic.

### I.4.2. Single-Core Droplets

The double flow-focusing chip used to make single-core double emulsions was selectively treated. The section is shown in dark grey on FIG. 2C was kept hydrophobic as the PFH was the phase in contact with the channel walls in this region.

To avoid the destabilization of the first emulsion it is therefore necessary to keep this area hydrophobic. The rest of the chip (in blue) was treated hydrophilic, because this is where water is in contact with the channel walls (1, 4, 5 on FIG. 4C). A flat PDMS layer was used as a substrate for the chip instead of a glass slide. Following the procedure developed by Bodin-Thomazo et al (2017) [N. Bodin-Thomazo, F. Malloggi, P. Guenoun, Marker patterning: a spatially resolved method for tuning the wettability of PDMS, RSC Adv. 7 (73) (2017) 46514-46519. doi: 10.1039/C7RA05654K], the hydrophobic region was selectively patterned using a black permanent marker (Stabilo Superfine), to prevent its activation during the subsequent plasma treatment. A mirror line of this marker patterning was drawn on the PDMS layer substrate. The chip and the substrate were put in an air plasma (18 W for 1 min), after which they were aligned to fit the marker pattern. 2 minutes after the bonding of the chip, methanol was passed through using the pressure controllers at 20 mbar for 2 minutes to remove the marker. Water was subsequently passed through for 30 minutes.

### I.5. Production of Multi-Core Double Emulsion Droplets

The multi-core double emulsions consist of three phases (FIG. 1, top left and bottom left):
The dispersed phase is made of water with 0.9% wt NaCl and 1% wt sodium fluorescein.
The intermediate phase is made of PFH or ATBC and 5% wt of Krytox 157 FSL.
The continuous phase is made of water and 0.1% wt of F₈TAC₁₃ (when PFH is used in the intermediate phase) or H₁₂TAC₇ (when ATBC is used in the intermediate phase).

First, a primary nanoemulsion of normal saline (0.9% wt NaCl in water) and fluorescein, in oil was produced by nanodroplets inside a larger oil droplet, surrounded by water. The pressures and flow rates used to produce these emulsions are shown in Table 1 below:

TABLE 1

| | | P (mbar) | Q (μL/min) |
|---|---|---|---|
| 20 μm | Φ_{d} | 180 | N/A |
| | Φ_{c} | 450 | 8 |
| 30 μm | Φ_{d} | 150 | N/A |
| | Φ_{c} | 200 | 2.5 |

### I.5.1. Production of Water Single-Core PFH Droplets

Single-core double emulsion droplets consist of three phases:
The dispersed phase is made of water with 0.9% wt NaCl and 1% wt sodium fluorescein.
The intermediate phase is made of PFH and 5% wt of Krytox 157 FSL.
The continuous phase is made of water and 0.1% wt of F₈TAC₁₃.

They were produced using the double flow-focusing junction device shown in FIG. 2C, where section 1 represents the channel where the dispersed phase enters; section 2 represents the channel where the intermediate phase enters; section 3 represents the channel in which the first emulsion of water in PFH is formed; section 4 represents the channel where the continuous phase enters; and section 5 represents the channel where the double emulsion of a single water droplet in PFH in water is formed. After the surface treatment, the different fluid phases were infected into the chip by applying a pressure of 20 mbar to the headspace of their respective inlets using the pressure controllers. The flow rates were adjusted to encapsulate exactly one water droplet in each double emulsion shown in Table 2:

TABLE 2

| | | P (mbar) | Q (μL/min) |
|---|---|---|---|
| 20 μm | Φ_{d1} | 60 | N/A |
| | Φ_{d2} | 50 | 1.0-1.5 |
| | Φ_{c} | 80 | 2.0-2.5 |
| 30 μm | Φ_{d1} | 65 | N/A |
| | Φ_{d2} | 50 | 0.9-1.3 |
| | Φ_{c} | 75 | 2.0-2.5 |

I.5.2. Production of Plain Tributyl o-Acetylcitrate Droplets

Plain ATBC single emulsions were made using a dispersed phase of ATBC and 0.025% weight Nile red. The continuous phase was made of water and 0.1% wt H₁₂TAC₇.

The droplets were made using the same chip geometry as the multi-core double emulsion droplets (FIG. 2A), where the dispersed phase was pinched by the continuous phase at the nozzle to produce droplets. The pressures and flow rates used are shown in Table 1.

### I.5.3. Characterization of Droplets

The size and polydispersity of the droplets were determined from a series of images taken with an ultra-fast camera (Model SC1, Edgertronic, USA) during their production.

The collected recordings were analyzed with an in-house MATLAB program. The code uses a Circular Hough Transform (CHT) based algorithm for locating the droplets and then estimating their diameter along with the polydispersity index (PDI).

The aqueous volume fraction in the single-core double emulsion droplets is calculated by monitoring two parameters: the number of droplets generated per second, which can be captured via the high-speed camera, and the flow rate of each phase recorded from the flow unit of the pressure controller. The volume fraction is then predicted using an in-house MATLAB code.

The size and the polydispersity of nanosize droplets in the multi-core droplets were determined by dynamic light scattering using an ALV/CGS-3 platform-based goniometer system (from ALV GmbH). The measurements were performed on emulsions diluted 1000 times, at room temperature, and scattering angles, θ, ranging from 60° to 130°, with a step of 10°. At each angle θ, the device provided the decay rate Γ_{θ}=q²(k_{B}T/6πηR); where k_{B} is the Boltzmann constant, T is the temperature in K, η is the viscosity of the solvent, and q(θ)=4πn sin(θ/2)/λ is the magnitude of the scattering vector. The refractive index of the solvent is n=1.33 and λ=633 nm is the laser wavelength.

A fit of the curve by the cumulant method thus made it possible to determine the hydrodynamic droplet mean radius R along with the polydispersity index (PDI) [A. G. Mailer, P. S. Clegg, P. N. Pusey, Particle sizing by dynamic light scattering: non-linear cumulant analysis, Journal of Physics: Condensed Matter 27 (14) (2015) 145102].

### I.6. Interfacial Tension

Three interfacial tensions between the aqueous and PFH phase were measured with an error of 2 mN/m using a Tracker tensiometer (Teclis, France) at 20° C. The tensiometer analyzes the shape of a pendant or rising drop in bulk, using the Young-Laplace equation to derive the interfacial tension. In the case where the measurement was taken between PFH and water with F8TAC13, or when it was taken between water and PFH with 5% Krytox, the pendant drop method was used [J. D. Berry, M. J. Neeson, R. R. Dagastine, D. Y. Chan, R. F. Tabor, Measurement of surface and interfacial tension using pendant drop tensiometry, Journal of Colloid and Interface Science 454 (2015) 226-237]. The water drop volume was kept constant at 2 mm3. It has been waited until the interfacial tension reached an equilibrium value to extract the interfacial tension value. In the case where the measurement was taken between PFH with 5% Krytox, and water with F₈TAC₁₃, another method was used. The bulk was PFH and 5% Krytox on the bottom, and water with F₈TAC at the top (because PFC is denser than water the interface between the two was stable). A rising water droplet was formed with a curved 0.6 mm diameter needle on a Hamilton 700 series syringe, in the PFH and Krytox phase, therefore covered in Krytox, and creamed to the interface, where it went halfway into the water with F₈TAC13 phase. The droplet remained stable at the interface for a few minutes before collapsing. The interfacial tension at top interface of the droplet, which mimics the inner droplet of a single-core double emulsion droplet in dewetting conformation, was measured. As a control test, we measured the interfacial tension of a sessile air bubble in water: a value of 72 mN/m was measured.

### I.7. Determination of ADV Threshold

To determine the ADV threshold (PADV) of PFH droplets, the intersect method has been used, it is described by Aliabouzar et al., Osborn et al., and Fabiilli et al. [J. Osborn et al., Acoustic Droplet Vaporization of Perfluorocarbon Droplets in 3D-Printable Gelatin Methacrylate Scaffolds, Ultrasound in Medicine & Biology 47 (11) (2021) 3263-3274; Aliabouzar et al. Effects of droplet size and perfluorocarbon boiling point on the frequency dependence of acoustic vaporization threshold, The Journal of the Acoustical Society of America 145 (2) (2019) 1105-1116; M. Fabiilli et al. The role of inertial cavitation in acoustic droplet vaporization, IEEE Transactions on Ultrasonics, Ferroelectrics and Frequency Control 56 (5) (2009) 1006-1017]. To summarize, the Fast Fourier Transform of the acoustic signal was analyzed and the magnitude of the subharmonic peak at 0.5 MHz, which is indicative of ADV, was normalized by the pressure. This value was plotted against the acoustic pressure. P_{ADV} was determined as the pressure at which the normalized magnitude starts to increase (2.1 MPa). As an alternative method, the probability of ADV occurring for each pressure was determined.

Every time a subharmonic peak at 0.5 MHz was observed, it was counted as one event. The number of events out of 100 pulses was counted to obtain a probability. It has been found that the pressure value at which the probability, p, is %, matched the P_{ADV} value obtained with the intersect method. This probability method was then used to determine the P_{ADV} of multi-core PFH droplets and single-core PFH droplets, as it is more precise and can be fitted with a probabilistic model.

I.8. Delivery of Fluorescent Probe from Droplets

### I.8.1. Preparation of the Sample

To remove free fluorescent probe from the sample, the droplet solution was pipetted into tubes with membrane size 100 kDa MWCO (Thermo Scientific, UK) to perform filtration. The sample was centrifuged, at 12 000 g for 40 min and 4° C. The filtered solution was removed and replaced with new bulk solution. This filtration through centrifugation step was repeated 5 times to achieve complete removal (until the filtered solution no longer contained fluorescent probe detectable with the spectrofluorometer).

For fluorescein release experiments, the sample was then prepared in 2 mL tubes (MC 200, Fisherbrand) containing 1 mL of normal saline (9 g NaCl/L of water) and 5 μL of droplet solution, taken from the bottom of the droplet sample tube where the droplets sedimented. For Nile red release experiments, the sample contained 1.5 μL of droplet solution, 200 μL of normal saline and 100 μL of ATBC. Since ATBC is less dense than water, it formed a layer above the water. Nile red from the droplets being hydrophobic, was released in the water but traveled to the oil layer.

### I.8.2. Acoustic Treatment of Sample

The signal consisted of sine-wave bursts at fundamental frequency 1.1 MHz. Duty cycles of 5% and 50% (meaning that the sample is insonified respectively 5% and 50% of the total time) were used, and a pulse repetition frequency of 11 kHz. Peak negative acoustic pressures varying from 0.2 Mpa to 2.3 Mpa were used. The total experiment time was 7 minutes, but the actual insonation time was either 0.35 or 3.5 minutes, and the temperature difference did not exceed 1.5° C. For every acoustically treated sample, 1 mL of normal saline was pipetted into the 2 mL centrifuge tube, the droplets were added and the sample tube was placed on the ultrasound set up for insonation. Two control samples were used at the beginning of each set of experiments. One control sample was used as a 100% release reference. The same volume of droplets solution was destabilized in 200 μL of methanol then 800 μL of normal saline was added. The second control was a sample of 1 mL of normal saline and the same volume of droplet solution as for the other samples. The passive release was assessed over 7 minutes, with no acoustic treatment.

### I.8.3. Detection of Fluorescence to Determine Release Percentage

The release was determined from the energy of fluorescence of the probe detected in the supernatant of the sample. For experiments with fluorescein, 850 μL of supernatant was pipetted into a 1 mm thick optical path spectroscopy cuvette (Hellma). The cuvette was placed in a spectrofluorometer (Jasco Spectrofluorometer FP-8300, Germany). The fluorescence was analyzed with an excitation wavelength of 470 nm and emission at 513 nm. To detect Nile red, 70 μL of the top ATBC layer in the sample was taken and pipetted into a 100 μL, 1 mm thick optical path spectroscopy cuvette (Hellma). The excitation wavelength was 530 nm and the emission wavelength was 568 nm. The concentration released was obtained from standard curves for each fluorescent probe. The concentration release was compared to the initial concentration in the droplets to obtain a percentage.

### I.8.4. Analysis of Release Data

For each sample, the pressure at which the experiment was performed was converted into an acoustic energy value. The acoustic energy at each pressure for each number of cycles was calculated as:

$$E = \frac{n < {P}^{2} >}{{ρ}_{o} c}$$

(1) where <P> is the average pressure integrated over one period, n is the number of cycles in one pulse, ρₒ is the density of water and c is the speed of sound in water.
The percentage of release was normalized by R³, where R is the radius of the droplet, and by the number of droplets in the focal zone, n_{f}.
The equation used to fit the data is:

$$\begin{matrix}f = β ( 1 - {e}^{- α E} ) & f = \frac{{C}_{B}}{{C}_{0} {R}^{3} {n}_{f}}\end{matrix} .$$

(2) where C_{B} is the concentration of probe molecules in the bulk, C₀ is the initial concentration in the droplets, R is the radius of the external droplet, n_{f} is the mean number of droplets in the focal zone,

$$β = \frac{\frac{4}{3} π}{{V}_{B}}$$

is the coefficient of the fit, α is a constant, and E is the acoustic energy. The coefficient of the fit, β is 6.25×10⁻⁷ μm⁻³.

### II. RESULTS

### II.1. ADV of Multi-Core and Single-Core PFH Double Emulsions

The PADV of the multi-core and single-core double emulsion PFH droplets are respectively 2.2 MPa and 1.8 MPa (FIG. 6).

II.2. More Efficient Release from Larger Droplets

The pressures at which release starts for both types of PFH droplets are below their respective P_{ADV} (Table 3):

TABLE 3

| | M [symbol] PFH | | | | | S [symbol] PFH | | | | M [symbol] [symbol] TBC | | Plain ATB [symbol] | | | |
|---|---|---|---|---|---|---|---|---|---|---|---|---|---|---|---|
| R (μm) | 20 | | | 30 | | 20 | | 30 | | 20 | | 20 | | 30 | |
| Nb. cycles | 5 | 20 | 50 | 5 | 50 | 5 | 50 | 5 | 50 | 5 | 50 | 5 | 50 | 5 | 50 |
| P [symbol] (MP [symbol] ) | 0. [symbol] | 0.2 | 0.2 | 0.2 | 0.2 | 0.5 | 0.2 | 0.2 | 0.2 | 0.5 | 0.5 | 0.8 | 0.2 | 0.5 | 0.2 |
| E (kW/cm [symbol] ) | 0.04 | 0.0 [symbol] | 0.07 | 0.0 [symbol] | 0.07 | 0.01 | 0.07 | 0.04 | 0.42 | 0.11 | [symbol] .07 | 0.04 | 0.07 | 0.04 | 0.07 |
| Start % | 2 | 2 | 4 | 2 | 4 | 11 | 18 | 16 | 6 | [symbol] | 18 | [symbol] | 2 | 6 | 2 |
| Max [symbol] % | 23 | 50 | 5 [symbol] | 80 | 88 | 6 [symbol] | 87 | 91 | 87 | 12 | 52 | 27 | 60 | 29 | 85 |

[symbol]

indicates data missing or illegible when filed

The percentage of release from 30 μm droplets is higher than from 20 μm droplets at all pressures. The pressures at which ATBC based droplets start to release their content and the maximum percentage of release are comparable to the ones for PFH based droplets of equal radius. Incorporating a hydrophilic dispersed phase in the core does not have an effect on the pressure at which release start and reduces the maximum release percentage by 8%. Overall, 30 μm droplets release a higher percentage of their content than 20 μm droplets at all pressures and for all droplet types, but the pressure at which release begins is not significantly different.

### II.3. Increasing the Acoustic Energy Increases the Release

ADV has been shown to occur during the first cycle of the first pulse of an acoustic signal, provided that the threshold pressure is achieved. Increasing the number of cycles, and thus the acoustic energy into the system (eq. 1) from 5-cycle pulses to 50-cycle pulses should not have any effect on the release, if the threshold pressure of ADV is reached. The percentage of release is normalized by R³ and n_{f}, where R is the radius of the droplet and n_{f} is the number of droplets in the focal zone, and it is plotted versus the acoustic energy (FIG. 3). An increase in the release is observed as the acoustic energy increases, thus confirming that there is no ADV. This behavior occurs for both PFH droplets and ATBC droplets, thus suggesting the same mechanism of release.

II.4 More Efficient Release from Single-Core Droplets Compared to Multi-Core Droplets

The release from single-core PFH droplets occurs more abruptly compared to multi-core droplets (FIG. 4). Similarly, to multi-core PFH droplets, close to 50% of the release occurs from both 20 μm and 30 μm droplets before the ADV threshold.

### III. CONCLUSION

The ultrasonic release of fluorescent molecules from droplets made of biocompatible oils has been studied. The droplets produced by microfluidics, are stabilized by surfactants and dispersed in water. The hydrophobic dye was sequestered in droplets of ATBC, while the hydrophilic one was either in many water nanodroplets or in one micro droplet, dispersed in the oil droplets of ATBC or PFH.

A 1.1 MHz wave causes the release of the dyes at pressures significantly lower than those necessary to vaporize the droplets acoustically (P_{ADV}≈2 MPa). Delivery starts at P=0.1P_{ADV}.

A pressure of about 0.3 MPa releases about 30% of dye after an ultrasound exposure of only 2.3 min. it has been observed that the temperature increase due to the wave never exceeded 1.5° C.

A diffusive model can explain the release from all droplet types.

One of the main advantages of the double emulsion according to the invention is to ensure that delivery of biological active substance is as efficient as possible. For example, when delivering an antibiotic mixture, one being hydrophilic and a second one being hydrophobic by using a pill, the antibiotic should be concentrated in a manner than an efficient dose of said mixture will pass the intestinal barrier to act in the body against bacteria. By using the double emulsion structure according to the invention, the antibiotic mixture can target directly the place where it is needed (e.g. directly at the lung level for Chronic obstructive pulmonary disease (also known as COPD) treatment) with a reduced amount of active substances than the one in the pill. The hydrophobic one is located in the droplet water and the second one which is hydrophobic is located in the biocompatible oil droplet.

1. A double emulsion structure composed of at least one water based droplet that comprises at least one first biocompatible amphiphilic molecule and at least one water soluble compound, wherein said water based droplet is included in an oily droplet that comprises at least one biocompatible oil and at least one second biocompatible amphiphilic molecule and wherein said structure allows the on-demand release of said water-soluble compound when subjected to low-frequency ultrasound.

2. The double emulsion structure according to claim 1, where the size of the oily droplet is comprised between 100 and 1000 nanometers.

3. The double emulsion structure of claim 1, where the size of the water-based droplet is comprised between 50 and 800 nanometers.

4. The double emulsion structure of claim 1, wherein the water-based droplet comprises a saline solution.

5. The double emulsion structure according to claim 4, wherein said saline solution has the plasma osmotic pressure and has a concentration of NaCl in water of approximately 0.5% to 1.5% wt.

6. The double emulsion structure of claim 1, wherein said at least one water soluble compound is a therapeutic active agent.

7. The double emulsion structure of claim 1, wherein said lipidic droplet comprises at least one biocompatible oil and at least one second biocompatible amphiphilic molecule.

8. The double emulsion structure of claim 1, wherein said biocompatible oil is selected in the group consisting of PFC or ATBC.

9. The double emulsion structure of claim 1, wherein the lipidic droplets comprise one or several water-based droplets and wherein the water-based droplets comprise one or several different therapeutic active agents.

10. The double emulsion structure of claim 1, wherein the therapeutically active agent is a chemical compound, a protein, a peptide, an antibody a DNA structure or an RNA structure.

11. The double emulsion structure of claim 1, wherein the structure releases said therapeutic active agent when it is subject to an acoustic signal at a frequency of 0.5 to 2 MHz over an insonation time comprised between 0.1 to 5 minutes.

12. The double emulsion structure according to claim 11 wherein the maximum peak negative pressures to release the content of the droplets are between 0.2 to 1 MPa.

13. The double emulsion structure of claim 1, wherein the lipidic droplet contains a hydrophobic therapeutical active agent.

14. The double emulsion structure of claim 1, wherein said structure is a liquid one or a lyophilized one.

15. A double emulsion comprising the double emulsion structure according to claim 1, in an aqueous continuous phase.

16. A method for on-demand release of the water-soluble compound comprising subjecting a double emulsion structure according to claim 1, to an acoustic signal at a frequency of 0.5 to 2 MHz over an insonation time comprised between 0.1 to 30 minutes.

17. Process of preparation of a double emulsion structure according to claim 1, wherein said double emulsion structure is generated in at least one microfluidic device using flow focusing geometries.

18. A Kit for treating a patient subject to a disease comprising a double emulsion structure according to claim 1 and a focused ultrasound device (FUS).
